(19) United States
(12) Patent Application Publication
(10) Pub. No.: US 2019/0343454 A1
(43) Pub. Date: Nov. 14, 2019

(54) Device for measuring and teaching human position and/or movement

(71) Applicant: MioCoach Oy, Helsinki (FI)

(72) Inventors: Arttu SUOKAS, Helsinki (FI); Ari MÄKELÄ, Espoo (FI)

(73) Assignee: MioCoach Oy, Helsinki (FI)

(21) Appl. No.: 16/475,712

(22) PCT Filed: Feb. 2, 2018
(86) PCT No.: PCT/FI2018/050076

§ 371 Date: Jul. 3, 2019

(30) Foreign Application Priority Data
Feb. 9, 2017 (FI) 20175112

(51) Int. Cl. A61B 5/00; A61B 5/11; G09B 19/00

(52) U.S. Cl. CPC A61B 5/486; A61B 5/1116; A61B 5/002; A61B 5/6833; A61B 2562/0261; G09B 19/003; A61B 2562/0257; A61B 2505/09; A61B 5/7455

(57) ABSTRACT

The invention relates to a device for measuring and teaching human position and/or movement, comprising an actuator, specifically a motor-gear assembly, a sensor for measuring a change of the human position and/or movement in at least one joint area, and a control unit for storing the position and/or movement changes measured by the sensor and for controlling the actuator. The device comprises a power supply for operating the actuator, a tensioning mechanism, which is attachable to human skin, linked to the actuator and operated by the actuator, and which communicates to the skin a correction-oriented tensioning stimulus.

The invention relates to a device as set forth in the preamble of claim 1 for measuring and teaching human position and/or movement.

Human ability to perceive his/her body and its movements has declined in everyday activities, working life and sports and, hence, various repetitive erroneous movement patterns and positions are often a cause of various exertion-related pain conditions and injuries, the effectiveness at work and the joy of leisure time activities diminishing with health care costs increasing. Typically, the training of human posture and especially movement is attended by a physical education or health care professional, for example a physiotherapist or a personal trainer, or by some other such expert who provides instructions for correcting erroneous movement patterns and/or positions. The prior art also discloses sitting posture training equipment, comprising a position sensor and a vibration mechanism in combination, which is only used for correcting the most typical error in sitting posture. Such equipment is based on vibration-induced stimulation which alerts the user to correct his/her position. The prior art further discloses various applications which measure a person's daily activities and, based on the measurements, provide a result indicating how large is a portion of the day that the person is sitting, standing, lying down and/or moving.

Publication EP3079581A2 discloses an assistive, flexible suit for assisting a person's mobility, which is particularly used for assisting a person's walking and gait movements. The suit comprises elastic strain gauges for measuring joint mobility, as well as actuators for generating desired forces in an effort to facilitate the movement of leg muscles. The suit enables measurements to be conducted continuously, and the actuator, which produces forces for muscular assistance, is adjustable on the basis of measuring results obtained from the strain gauges.

Publication IN201611025988A discloses a strain gauge, which is used for measuring human muscle contraction, as well as for gathering data from muscle measuring results.

Publication JP2016127898A discloses a strain gauge, particularly for measuring a correct foot position in connection with various physical exercises.

The prior art devices do not, however, enable versatile and multifunctional assistance to people in terms of perceiving the position and movements of their own body in such a way that various pain conditions and injuries caused by repetitive erroneous movement patterns and positions in everyday activities, working life and sports could be corrected in a versatile and effective manner, even without the assistance of a physical education or health care professional.

It is an objective of the invention to solve the foregoing problems and drawbacks, as well as to provide a device for measuring and teaching human position and/or movement, wherein the problems of systems known from the prior art will have been eliminated or at least minimized, especially in terms of versatility.

In order to attain the foregoing and subsequently presented objectives, the device according to the invention for measuring and teaching human position and/or movement is principally characterized by what is set forth in the preamble section of claim 1.

Further preferred characterizing features and applications of the invention are presented in the dependent claims.

A device of the invention is provided for measuring and teaching human position and/or movement, comprising an actuator, a sensor for measuring a change of the human position and/or movement in at least one joint area, and a control unit for storing the position and/or movement changes measured by the sensor and for controlling the actuator, and said device comprising a power supply for operating the actuator, a tensioning mechanism which is attachable to human skin, linked to the actuator and operated by the actuator, and which communicates to the skin a correction-oriented tensioning stimulus.

A method is provided for measuring and teaching human position and/or movement, said method comprising the use of a device, which comprises an actuator, a sensor for measuring a change of the human position and/or movement, and a control unit for storing the changes of the sensor-measured position and/or movement and for controlling the actuator, the device used in said method comprising a power supply for operating the actuator, a tensioning mechanism for the actuator which, on the basis of measuring results obtained from the sensor, generates a tensioning stimulus in the corrective direction for correcting the human position and/or movement according to an instruction supplied by the control unit.

According to one preferred further characterizing feature of the invention, the sensor is placed in connection with an elastic, tape-like member attachable to human skin.

According to one preferred further characterizing feature of the invention, the tensioning mechanism is disposed in connection with an elastic, tape-like member.

According to one preferred further characterizing feature of the invention, the device control unit comprises electronics and/or a processor for operating the actuator, such as a motor-gear assembly, and for gathering measurement data, as well as preferably a communication link to a mobile application for giving instructions to the control unit and for regulating operation of the control unit, as well as for receiving the measurement data.

According to one preferred further characterizing feature of the invention, the device comprises a wireless transmitter, such as a Bluetooth transmitter, for sending measurement results of the sensor to the control unit. According to one preferred further characterizing feature of the invention, the device comprises a pulling part disposed at one end of the elastic tape-like member and a head disposed at the other end, and the device further comprises a motor-gear assembly, which is coupled to the sensor mounted on the head and which is meshed by way of a gear with a toothing provided on a center disc disposed on the head, a reel which is disposed concentrically with the center disc and which encircles the center disc, and on which reel is provided a length of pullstring, a torsion spring disposed inside the center disc and having one end supported in connection with the reel and the other end meshed with the toothing of the center disc, and one end of the pullstring is in attachment with the pulling part and the other end on the reel. According to one preferred further characterizing feature of the invention, it is for the pullstring that the tape-like member is preferably provided with a groove, a pathway, or the like formation.

According to one preferred further characterizing feature of the invention, the device further comprises an operating button for setting an action point for the device.

According to one preferred further characterizing feature of the invention, the device controlling unit comprises a means for establishing a wireless communication link to a separate gadget used for controlling the device by way of an application, for example a mobile application.

According to one preferred further characterizing feature of the invention, the device is attached by way of a tape-like member on top of a human joint area, the joint is set in a desired position and the device is calibrated on this point, whereby, when the position of a user's joint area changes in such a way that the skin stretches across the calibration point, a tensioning stimulus generated by the device's tensioning mechanism will be activated and a conscious and/or unconscious correction will take place as the user corrects the joint position as desired.

The device according to the invention for measuring and teaching human position and/or movement is a device, which measures incorrect movements of a human body and teaches a correct movement pattern, and which comprises an aggregate made up of a sensor and an actuator for activating a user's muscles and thereby for measuring and teaching the user's incorrect position and/or movement inflicting pain conditions and injuries. With a device of the invention, the position and/or movement of a human being will be corrected by activating the user's own muscles.

One preferred exemplary embodiment of the device according to the invention for measuring and teaching human position and/or movement by activating a person's own muscles comprises a tape-like, elastic member, which comprises a sensor for measuring the position and/or movement, said tape-like member being attached to the person's skin across a human joint area. The device according to this exemplary embodiment of the invention is a device, which measures and teaches a human body's position and/or movement, and which is adapted to be attached with a tape-like member to the skin on top of a desired joint area. In case of losing control over a position and/or movement, the device applies to the skin a tensioning stimulus in a position- and/or movement-correcting direction, whereby the position and/or movement correction occurs consciously with a user reacting to the stimulus as well as unconsciously by way of a reflex arc. Hence, the device according to this exemplary embodiment of the invention is a device attachable to the skin for measuring and teaching human position and/or movement, which device measures the position and/or movement of a joint area through changes in the skin length and, once adjusted values are fulfilled, applies to the skin a tensioning stimulus in a desired direction. This stimulus reminds the user on conscious level that an error has occurred, whereby the user can make a conscious correction regarding for example forward head postures when working at a computer terminal. In addition to consciously performed correction, according to the theory of proprioception, the stimulus initiates the unconscious correction of proprioceptive movement or position via a so-called reflex arc, the tensioning stimulus being communicated by way of a sensory nerve to the spinal cord, whence further to motor nerves which initiate position- or movement-correcting muscle activation. Proprioception refers to the sense of position and movement and relates to sensory information about position and movement of the limbs, trunk, and head. In proprioception, the human body converts a mechanical stimulus applied to the skin into a neural signal which is conveyed to the central nervous system, the body responding with a motor impulse for adjusting the position and movement.

One preferred embodiment of the device according to the invention comprises an adhesive surface provided by a tape-like member, a sensor, a tensioning mechanism, a wireless transmitter, such as a Bluetooth transmitter, a data processing unit or application, and a power supply. The sensor measures a change of the skin length and communicates the measurement result about changes in the skin length to a terminal device. Upon the fulfillment of desired threshold values set in the device control unit or application, the tensioning mechanism of the device activates, thus communicating to the skin a tensioning sensation which the person's central nervous system processes and transmits to the muscles an instruction to activate in the direction of tension, whereby the position-correcting movement occurring in the joint takes place as a result of the user's own muscle becoming activated. The force generated by the device's tensioning mechanism does not in itself produce, limit or coerce externally the joint movement but, instead, activates a central nervous system-related position/movement correction, the device enabling a person to unlearn incorrect positions and/or incorrect movement patterns.

The device according to the invention measures and practices body control, making it useful for measuring the time spent in incorrect positions and the incorrect movement of a joint, and for unlearning overstressing and/or incorrect positions and movement patterns.

The device according to the invention comprises a sensor, which is preferably disposed in engagement with an elastic, tape-like member attachable to the skin. It is the sensor included in a device of the invention that enables position changes in any joint area to be measured.

The device according to the invention comprises a tensioning mechanism, which communicates to the skin a tensioning sensation in a position- and/or movement-correcting direction, said tensioning sensation enhancing the proprioception or kinesthetic sense of a body, whereby the human body responds to proprioception with a motor impulse for correcting the position and/or movement.

The device according to the invention comprises also a control unit, such as a data processing unit or application, for storing sensor-measured position and movement changes and for adjusting individual threshold values and stimulus. According to one preferred embodiment of the invention, the control unit is a mobile application.

In a device of the invention, the sensor for measuring a change in position and/or movement may also be a stretch sensor, wherein the change in length is obtained by measuring the change in electrical conductivity caused by a change in the length of an electrically conductive material, for example a fabric or rubber or some other material, or a proximity/position sensor used for measuring a distance between two points by means that are optical or mechanical or involve the use of magnetism, electrical conductivity, sound or radio waves.

In a device of the invention, the tensioning mechanism for generating a tensioning stimulus (a tensioning sensation communicated to the skin and activating a conscious and/or unconscious correction of position or movement) can also be engineered by magnetism-related means, for example according to the operating principle of a solenoid, or by a change in the length of a muscle wire type material as the material is supplied with electric current, or by a shape memory alloy or other artificial, synthetic muscle solutions, or by other mechanical solutions.

It is by way of the device according to the invention, upon the fulfillment of individual threshold values set thereby, that there is generated a stimulus enhancing the sense of position and movement, whereby the body responds to the stimulus with a motor impulse for adjusting the position and movement. Hence, by means of a device of the invention there is provided a training intervention for achieving the conscious and/or unconscious learning of positions and movement patterns. After the training period, the body recognizes a desired position or movement pattern and the achieved result is an improvement of movement skill and ergonomics.

The device according to the invention is applicable as an ergonomic training and assessment method, which is used for example in workplace ergonomics and which comprises an analysis of measurement results, whereby the measurement results can be used as a basis for assessing the realization of ergonomic working positions and movements and the improvement of ergonomics in working positions and movements achieved by training carried out with the device.

The device according to the invention is applicable for example in physiotherapy for measuring body-stressing movement patterns and positions and for controlling a correct performance technique especially in home training and, in addition, it is possible to measure the result of a physiotherapeutic process by means of follow-up measurements.

The device according to the invention is applicable for example in the training of various sport disciplines for measuring and teaching discipline-specific movement patterns, as well as for tracking the development of discipline-related technique, and thereby also for measuring the result of a training process.

Accordingly, the invention can be used for measuring and training workplace ergonomics, for enhancing rehabilitation, as well as for improving movement control in everyday activities and sport performance. Users are typically people with a need of improving body positions or movement patterns, for example people suffering to various degrees from musculoskeletal problems, people in need of improving or measuring work ergonomics in connection, among others, with sedentary work, standing work, lifting work, work comprising repetitive monotonic movement patterns, people who require rehabilitation because of trauma or pathology in connection, among others, with motor rehabilitation following a spinal cord injury or a stroke, as well as athletes wishing to measure and define body control. When utilizing the invention, the work ergonomics, motor learning, rehabilitation, sport performance, work endurance and work efficiency will be improved while illness-related absences, repetitive strain injuries, sport injuries, and health care costs will be reduced.

The invention provides a possibility of measuring and teaching the position and movement control of any human joint area, and the stimulus generated by the invention, reminding the person of an incorrect position or movement, aligns itself naturally with a corrective motion direction as a result of which the user corrects the position consciously as well as unconsciously.

The invention will now be described in more detail with reference to the accompanying drawing figures whose details are nevertheless by no means intended to limit the invention strictly therein.

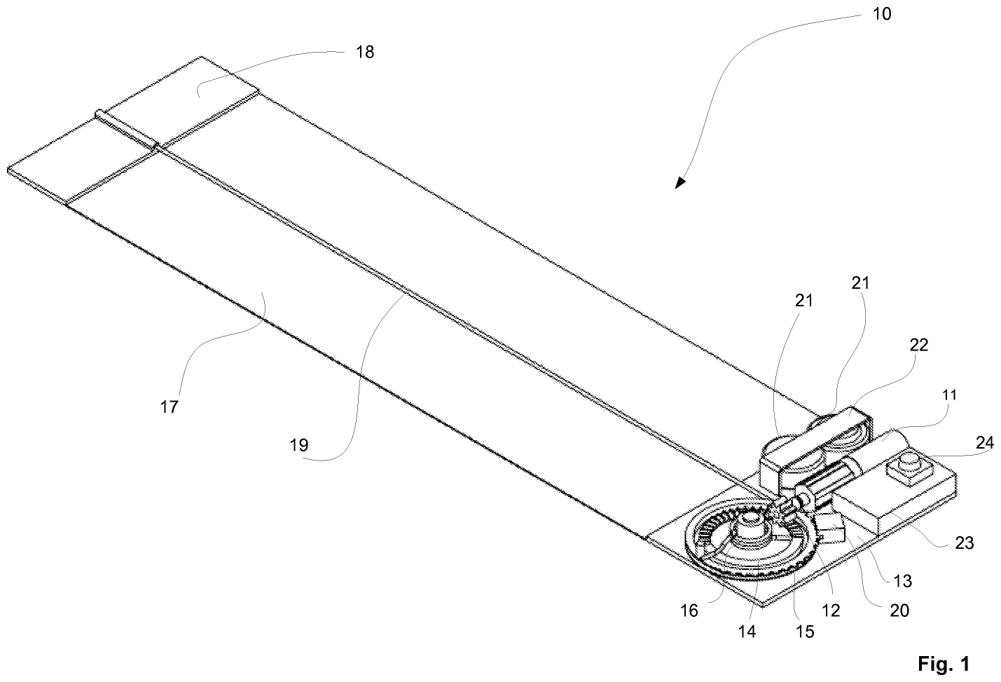

FIG. 1 shows schematically one preferred example for a device of the invention.

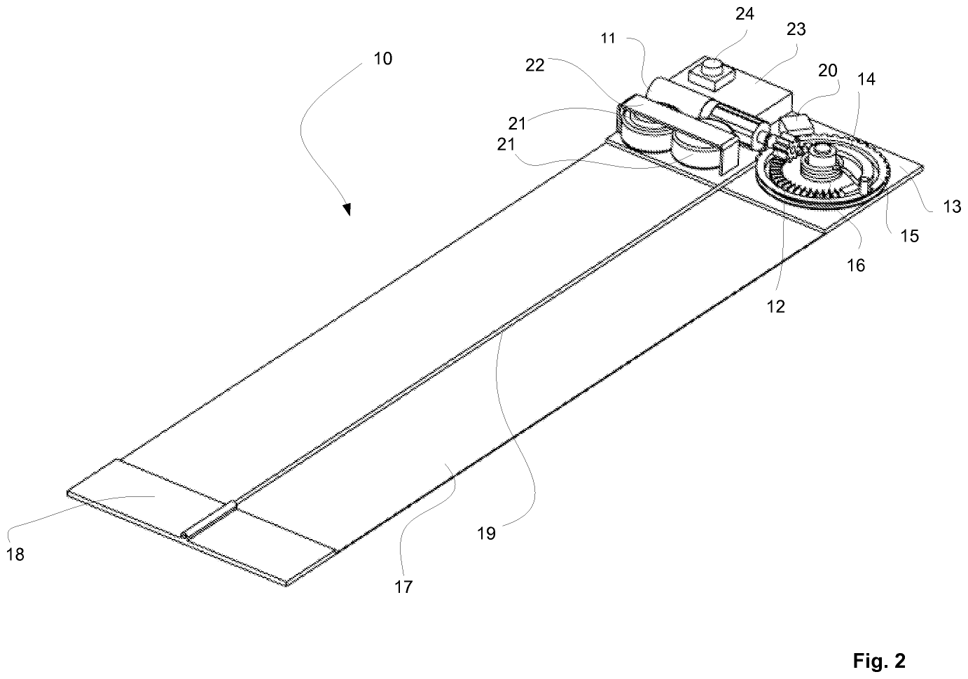

FIG. 2 shows schematically the example of FIG. 1 from another angle of view.

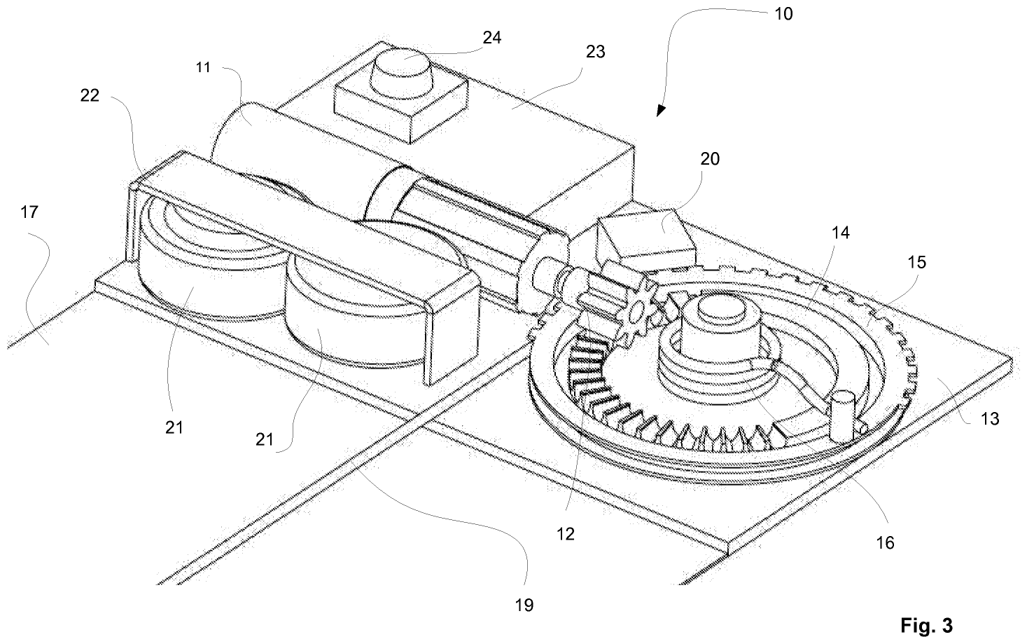

FIG. 3 shows schematically one so-called actuator end for the example of FIG. 1 in an enlarged partial view.

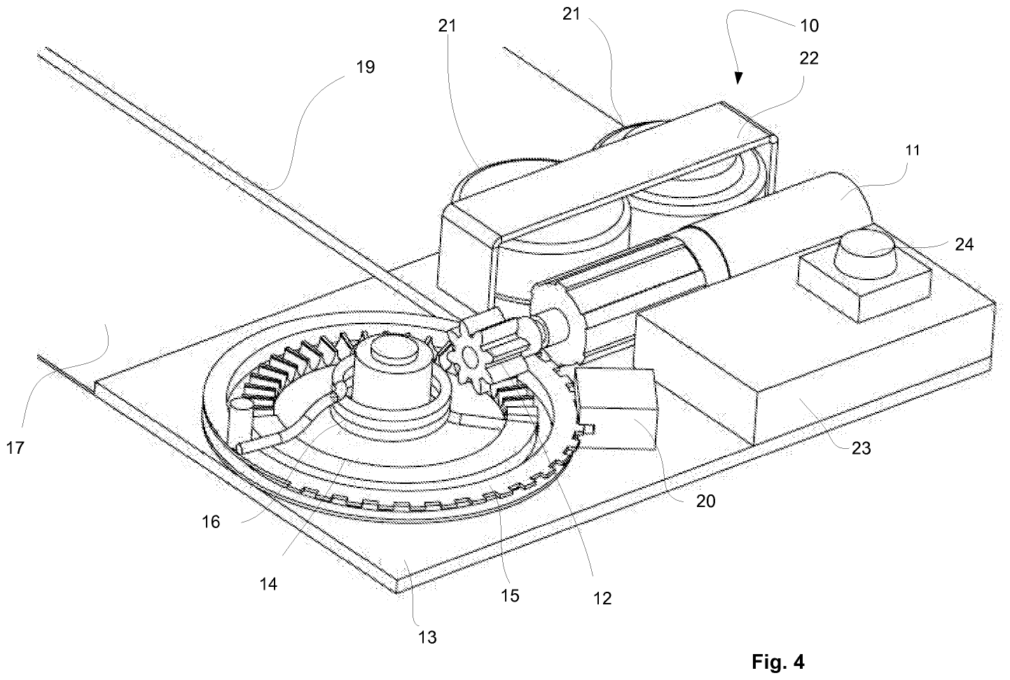

FIG. 4 shows schematically the partial view of FIG. 3 from another angle of view.

In the subsequent description and associated figures, like reference characters are used for denoting like components and subassemblies unless otherwise mentioned.

In the example presented in FIGS. 1-4, the device 10 for measuring and teaching human position and/or movement comprises an elastic, tape-like member 17 whose one end is provided with a pulling part 18 and whose other end comprises a head 13. The device 10 comprises a sensor 20 mounted on the head 13, a motor-gear assembly 11 coupled thereto and being in turn meshed by way of a gear 12 with a toothing provided on a center disc 14. The center disc 14 is rotatable and comprises, at least over part of its circumference, a toothing provided on the top surface and having the toothing of the gear 12 meshed therewith. Concentrically with the center disc 14 is arranged a reel 15, which encircles the center disc and on which reel 15 is provided a pullstring 19. Inside the center disc 14 is concentrically disposed a torsion spring 16, which in the exemplified device 10 is a coil spring whose one end is supported in connection with the reel 15, for example on a pivot member provided on a top surface of the reel 15 as shown in the figures, and whose other end is meshed with the toothing of the center disc 14. The elastic tape-like member 17 is preferably made of kinesiologic tape or some other tape-like material. Lengthwise in connection with the tape-like member 17 is provided a pullstring 19 whose one end is in attachment with the pulling part 18 and the other end on the reel 15. For the pullstring 19, the tape-like member 17 is preferably provided with a groove, a pathway, or the like formation along which the pullstring 19 is adapted to travel. The head 13 is also provided with batteries 21 as a power supply for the device 10, as well as with a battery holder 22. The head 13 has also mounted thereon a control unit 23 for the device 10 and an operating button 24 for setting an action point for the device. The device control unit 23 may also comprise means for establishing a wireless communication link to a separate gadget, for example to a mobile phone, a telephone, a computer or the like gadget, which is used for controlling the device 10 by way of an application, for example a mobile application. The separate gadget, such as a mobile phone, comprises preferably also a processing and storage means for measurement results.

The actuators for the device 10 comprise a motor-gear assembly 11, a tensioning mechanism 12, 14, 15, 16, electronics in the control unit 23, a power supply 21, as well as a wireless communication link such as Bluetooth, and an operating button 24. The pulling elements for the device 10 comprise a pullstring 19, a pullstring groove provided on the tape-like member 17, and a pulling part 18. The tape-like member 17 is an elastic, preferably replaceable tape for attaching the device 10 to the skin. Preferably, the control unit 23 is connected to a mobile application for the user's information, as well as for making more precise adjustments and gathering measurement data.

The device 10 has its sensor 20 measuring a position of the motor 11. The device 10 has its motor 11 rotating the reel 15 which contains the pullstring 19. The pullstring 19 travels freely in the tape-like member's 17 pullstring groove and is in attachment with the pulling part 18. The pulling part 18 attaches to the tape-like member 17. The device 10 has its control unit 23 regulating the operation of the motor 11 and collecting measurement results from the sensor 20 to forward the same to an application, e.g. to a mobile application via a Bluetooth link. The motor 11 rotates the reel 15 which tensions the pullstring 19, creating thereby traction between the head 13 and the pulling part 18 of the device 10 and further generating on the kin a stimulus in the direction of shortening. The device 10 does not move the joints mechanically but, instead, its action is based on enhancing the activation of a human body's own system achieved by the tensioning effect. When the motor 11 is not running, the reel 15 is able to rotate against the spring 16 as per elongation of the tape-like member 17. The sensor 20, preferably a pulse sensor, supplies the control unit 23 with position data, the motor 11 being supplied with a control instruction as an incorrect position is detected. The motor 11 displaces one end of the spring 16, tensions the spring 16 and the pullstring 19 until a core of the spring 16 diminishes against the center pivot and tensions the pullstring 19 for a desired period of time. From the sensor 20 is obtained a message about the success of correction and, as the motor 11 is driven back to a starting position, measuring an elongation of the tape-like member 17 is continued.

When the device 10 is employed, it is with a replaceable elastic tape present on the bottom of the tape-like member 17 that the device 10 is attached to the skin on top of a joint area to be exercised. The joint is set in a desired position, e.g. in a middle position, and the device 10 is calibrated on this point either by pressing the operating button 24 or by way of a mobile application. The stimulus activation delay, duration and rhythm for the device 10 can be regulated with the control unit 23 or with an application, e.g. a mobile application, connected thereto. The stimulus can be adjusted to activate immediately upon crossing a desired limit, whereby the correction of a movement and/or a position takes place immediately, or, on the other hand, the stimulus may become activated after a desired period, for example several minutes, from the occurrence of an incorrect position. The more rapid response is good for training movement control for example in sports while the delayed response is more suitable e.g. for training a more static position. When the position of a user's joint area changes in such a way that it crosses a set threshold value in an undesirable direction, whereby the skin stretches across the calibration point, a tensioning stimulus generated by the tensioning mechanism of the device 10 will be activated and the conscious and/or unconscious correction takes place and thereby the user corrects the joint to a desired position, for example a middle position.

The device 10 has its control unit 23 comprising electronics and/or a processor for operating the actuator 11, such as a motor-gear assembly, and for gathering measurement data, as well as preferably a communication link to a mobile application for giving instructions to the control unit 23 and for regulating operation of the control unit 23, as well as for receiving the measurement data. The device 10 has the application, for example a mobile application, of its control unit 23 gathering information about position changes over a desired time period as the device 10 is in service. The collected information can be compiled for reports of a desired type as necessary.

The device 10 may comprise not only a pulling part 18 disposed at one end of the tape-like member 17 but also several pulling parts placed longitudinally of the tape-like member 17 at a distance from each other.

A power supply of the device 10 and a separate control unit, which is intended for gathering data, can be connected to each other not by a wireless communication link but by way of cables. The control unit 23 of the device 10 comprises, among others, a piece of information regarding a desired joint position. It is by way of the control unit 23 that the tensioning stimulation can be switched off for measuring variations in the position and respectively switched on for training position or movement control. The stimulus actualizes as the position of a user changes across a designated level with the motor 11 drawing the pullstring 19 onto the reel 15 and bringing the pulling part 18 closer to the head 13, thus tensioning the tape-like elastic member 17. The sensor 20, which is measuring the motor's 11 position, gathers data about the motor's 11 position, conveying the position data to the control unit 23 which regulates activation of the motor 11.

According to one preferred embodiment, around the pullstring 19 is disposed a protection sleeve.

The tape-like member 17 consists preferably of an elastic tape-like material, for example kinesio tape, which is also known as kinesiology tape and which is used for bracing e.g. joint areas. As for its material, it typically consists of elastic cotton tape whose acrylic adhesive surface holds the tape immovably on the skin. Kinesio tape is used for the treatment of athletic injuries and various pains, most commonly for the rehabilitation of athletic injuries and for the alleviation of muscle pains. The tape provides support and ensures a correct motion path without disrupting blood circulation. Kinesio tape is often used also in efforts to prevent athletic injuries as it is possible thereby to provide direction and support for the motion path of a joint and to avoid harmful extreme positions.

1. A device for measuring and teaching human position and/or movement, said device comprising an actuator, a sensor for measuring a change of the human position and/or movement in at least one joint area, and a control unit for storing the position and/or movement changes measured by the sensor and for controlling the actuator, said device comprising a power supply for operating the actuator and a tensioning mechanism linked to the actuator and operated by the actuator, wherein the device further comprises an elastic, tape-like member attachable to human skin, a pulling part attached to the elastic, tape-like member and a pullstring in attachment with the pulling part, that the sensor for measuring the change of the human position and/or the movement in at least one joint area is placed in connection with the elastic, tape-like member, that the tensioning mechanism is disposed in connection with the elastic, tape-like member and that the tensioning mechanism for the actuator which, on the basis of the measuring results obtained from the sensor, generates on basis of measuring results obtained from the sensor a tensioning stimulus in corrective direction for correcting the human position and/or movement according to an instruction supplied by the control unit not in itself producing, limiting or coercing externally the change of the human position and/or the movement in the joint area but, instead, activating a central nervous system-related position/movement correction.

2. The device according to claim 1, wherein the control unit comprises electronics and/or a processor for operating the actuator, which is a motor-gear assembly, and for gathering measurement data, as well as preferably a communication link to a mobile application for giving instructions to the control unit and for regulating operation of the control unit, as well as for receiving the measurement data.

3. The device according to claim 1, wherein the device comprises a wireless transmitter, such as a Bluetooth transmitter, for sending measurement results of the sensor to the control unit.

4. The device according to any of claim 1, wherein the device comprises the pulling part disposed at one end of the elastic, tapelike-member and a head disposed at the other end, that the device further comprises the motor-gear assembly, which is coupled to the sensor mounted on the head and which is meshed by way of a gear with a toothing provided on a center disc disposed on the head, a reel which is disposed concentrically with the center disc and which encircles the center disc, and on which reel is provided a length of pullstring, a torsion spring disposed inside the center disc and having one end supported in connection with the reel and the other end meshed with the toothing of the center disc, and that one end of the pullstring is in attachment with the pulling part and the other end on the reel.

5. The device according to claim 4, wherein it is for the pullstring that the elastic, tape-like member is preferably provided with a groove, a pathway, or the like formation.

6. The device according to claim 1, wherein the device comprises an operating button for setting an action point for the device.

7. The device according to claim 1, wherein the device controlling unit comprises means for establishing a wireless communication link to a separate gadget used for controlling the device by way of an application, for example a mobile application.

8. The device according to claim 1, wherein the sensor is a stretch or proximity or position sensor.

9. The device according to claim 1, wherein the tensioning mechanism is one based on magnetism or based on a change in the length of a material or based on a shape memory alloy or based on a synthetic muscle solution.
